(19) United States
(12) Patent Application Publication
(10) Pub. No.: US 2010/0113919 A1
(43) Pub. Date: May 6, 2010

(54) Catheter arangement for insertion into a blood vessel for minimally invasive intervention

(76) Inventor: MICHAEL MASCHKE, LONNERSTADT (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/611,158

(22) Filed: Nov. 3, 2009

(30) Foreign Application Priority Data
Nov. 3, 2008 (DE) 10 2008 054 297.0

(51) Int. Cl. A61B 5/05; A61B 17/22; A61B 8/00

(52) U.S. Cl. 600/424; 606/159

(57) ABSTRACT

A catheter arrangement for insertion into a blood vessel is proposed. The catheter arrangement has a catheter with a proximal catheter tip, in which an intervention tool is guided to remove a blood clot from the blood vessel. The intervention tool has an element for trapping a blood clot, in particular a spiral, in the region of its tip. With a view to minimizing x-ray radiation during the treatment and safe guidance of the intervention tool a position identification element is disposed in the region of the catheter tip.

A clean copy of the abstract that incorporates the above amendments is provided herewith on a separate page.

### CROSS REFERENCE TO RELATED APPLICATIONS

This application claims priority of German application No. 10 2008 054 297.0 filed Nov. 3, 2008, which is incorporated by reference herein in its entirety.

### FIELD OF THE INVENTION

The invention relates to a catheter arrangement for insertion into a blood vessel, a medical examination and treatment facility with such a catheter arrangement, and a method for minimally invasive intervention at a blood vessel in the brain.

### BACKGROUND OF THE INVENTION

In modern medicine minimally invasive intervention tools are frequently used to eliminate blood clots or thrombi in vessels. When the blood clot is present in a blood vessel in the brain, the patient suffers an ischemic stroke. During an ischemic stroke the supply of blood to the brain is impeded by the blood clot, causing nerve cells to die. Since around three-quarters of all stroke patients suffer an ischemic stroke, a safe and fast treatment method is medically very important.

An intervention tool for removing a thrombus is known from US 2005/0033348, being guided to the thrombus with the aid of a microcatheter. At its proximal end the intervention tool has an extending tip to collect the thrombus. When the blood clot is withdrawn a balloon is inflated downstream at the catheter, so that the catheter sits finely in the blood vessel and the blood flow does not disrupt the minimally invasive surgical intervention. One disadvantage of the treatment described in the above-mentioned document is however that a continuous x-ray examination has to be carried out, some of the time using contrast agent, exposing the patient to radiation, to observe the insertion, advance and removal of the microcatheter in the patient.

### SUMMARY OF THE INVENTION

The object of the invention is to specify a catheter arrangement for inserting an intervention tool into a blood vessel to remove a blood clot, which allows safe guidance of the intervention tool while minimizing x-ray radiation during the treatment.

According to the invention the object is achieved by a catheter arrangement for insertion into a blood vessel, comprising a catheter with a proximal catheter tip, in which an intervention tool is guided to remove a blood clot from the blood vessel, having an element for trapping a blood clot, in particular a spiral, in the region of its tip, with a position identification element disposed in the region of the catheter tip.

The invention is based on the consideration that safe guidance of the intervention tool with the minimum x-ray radiation required for imaging is allowed in that the inserted, proximal end of the catheter is provided with a position identification element, so that the location of the catheter is visualized and checked. This facilitates catheter navigation considerably so that few x-ray recordings are required during the treatment.

The catheter tip is tracked and navigated in the blood vessel by showing the position identification element overlapping with a representation of the blood vessel. The representation is a representation of the blood vessel recorded beforehand by means of a medical imaging modality, for example an x-ray system, and reconstructed. Alternatively the position identification element can be located by imaging the interior of the blood vessel. After spatial calibration with the imaging modality, the position data of the position identification element can be overlaid on an x-ray image in 2D, 3D or 4D, so that x-ray recordings only have to be taken at particularly critical points. It is thus possible for a treating physician to track its continued movement in the blood vessel, in particular over the entire route up to the thrombus, on a screen. The position identification element is in particular configured to supply information about its position from the interior of the blood vessel continuously or at short time intervals of a few seconds or fractions of a second.

The position identification element can be configured here such that the position of the proximal end of the catheter is identified “from outside”, thus providing location information relating to the movement of the intervention tool. Alternatively the position identification element can transmit images from the interior of the blood vessel, which are used to verify the position of the catheter or intervention tool, as branches and curves of the blood vessel for example can be clearly identified both in the visual images and also in the reconstruction of the blood vessels. In this instance the topography of the inner wall of the blood vessel is determined and taken into account during navigation of the catheter.

The position identification element is preferably part of a location system. The location system is set up in particular to allow the position identification element to be located in all three spatial directions. The location system is used in particular to determine both the absolute and the relative position of the position identification element in relation to the blood clot for example. The position data obtained by the location system facilitates the safe insertion of the catheter and its navigation to the target region. The position data can also advantageously be included in the computational correction of motion artifacts and the like.

The location system preferably comprises a position transmitter and a position receiver, one of which is the position identification element. The position identification element can be both the position transmitter and also alternatively the position receiver. In addition to the position identification element a corresponding receiver or transmitter is provided outside the patient. At least one transmitter can be associated, as a position identification element with emission in all three spatial directions, with an external receiver, which is located in proximity to the patient. Conversely the catheter can support a receiver with X,Y,Z receive directions, which is associated with an external transmitter, to allow spatial location of the catheter tip and thus of the intervention tool.

With a view to particularly efficient minimally invasive intervention the position identification element is preferably disposed at the tip of the intervention tool. The tip of the intervention tool can thus also be located in real time in relation to the 3D reconstructions of the blood vessels when collecting and removing the blood clot.

Advantageously the location system is optionally an electromagnetic location system or an ultrasound system. An electromagnetic location system, wherein electromagnetic signals are used to determine the position of the end of a guide wire in a living being, is known from DE 10 2004 058 008. In such an electromagnetic location system electromagnetic receive or transmit coils are positioned on the intervention tool, to communicate with corresponding external electromagnetic transmit or receive antennas. Such a location system is able to locate the tip of the intervention tool very precisely, allowing the current position of the intervention tool to be superimposed on medical images, thereby considerably facilitating navigation of the intervention tool. Alternatively the location system is an ultrasound system. In DE 198 52 467 A1 a catheter tracking system for locating a catheter head based on ultrasound measurements is described. Such a method is also known as sonomicrometry and is based on finding the distances between miniature omnidirectional ultrasound transducers by measuring the time required for the ultrasound signals to travel the distance between the ultrasound transducers, this then being multiplied by the speed of sound.

According to one preferred variant the position identification element is an imaging sensor. The imaging sensor can be used to transmit “live images” from the site of the minimally invasive intervention to an externally located playback facility, e.g. a computer-controlled visualization system with a connected monitor. The insertion and guidance of the catheter into and through the vessels and the specific positioning of the intervention tool can be tracked with checks in real time. Such a high-resolution location representation allows fine corrections of the position of the catheter within a narrow time frame.

The imaging sensor is advantageously configured and aligned so that its field of vision covers a spatial region around the catheter tip and/or a spatial region in front of the catheter tip. In other words the imaging sensor “looks” radially outward, depending on the specific arrangement and/or depending on the type and operating principle of the sensor and/or depending on the material of the intervention tool in some instances also “through” the intervention tool or past it. Alternatively the field of vision of the imaging sensor primarily covers the spatial region in front of the catheter tip, in other words “looks” forward relative to the direction of insertion of the catheter, which is particularly expedient during the injection process, as well as for monitoring the process of inserting the catheter and its advance. Optimally the two possibilities mentioned above are suitably combined for the imaging sensor, so that the sensor has the greatest possible field of vision both in a radial and forward direction.

According to one preferred embodiment a transparent window is provided in the region of the catheter tip. A transparent window here is understood to mean a region at the proximal end of the catheter, which is permeable for the beams, with which the imaging sensor operates. In the simplest instance the region extends around the periphery of the catheter. The transparent window is configured so that imaging by means of the imaging sensor is possible at least in the radial direction but preferably also in the axial direction.

The possibilities for imaging by means of the optical sensor are extended in that according to a further preferred embodiment the imaging sensor can be moved out of the catheter. For example provision can be made for the sensor to be moved out of a “retracted” stop position in proximity to the proximal end of the catheter in a forward direction from the catheter to define an observation point with varying positions, from which regions further ahead can be inspected, while keeping the position of the catheter constant. To this end the imaging sensor can be disposed for example on a micro or inner catheter that can be displaced relative to the catheter and is disposed in its hollow chamber, or on an inner part. An opening for the imaging sensor is provided here on the proximal end face of the catheter, said opening being adequately sealed so that no fluids can penetrate into the interior of the catheter, regardless of whether the imaging sensor is in the retracted or extended position.

An (acoustic) ultrasound sensor is preferably provided as the imaging sensor. Ultrasound imaging (sonography) takes place according to the so-called echo-pulse method. An electrical pulse of a high-frequency generator is converted to a sound pulse in the probe of an ultrasound transducer (generally a piezo crystal but can also be a silicone-based sensor) and transmitted. The sound wave is scattered or reflected partially or completely at the non-homogeneities of the tissue structure. A returning echo is converted to an electrical signal in the probe and then visualized in a connected electronic evaluation and display unit, during which process a 2D or 3D scan of the examination region can be performed by swiveling the sensor mechanically or electronically. Intravascular ultrasound imaging (IVUS) is particularly suitable for imaging deeper tissue layers and vessel structures.

According to a second advantageous variant a magnetic resonance sensor is provided as the imaging sensor. This is a so-called IVMRI sensor for intervascular magnetic resonance tomography (IVMRI=Intra Vascular Magnetic Resonance Imaging). During magnetic (nuclear) resonance tomography the magnetic moments (nuclear spins) of the atomic nuclei of the examined tissue are aligned in an external magnetic field and excited to precession by radio waves that are radiated in, with an electrical magnetic resonance signal being induced as a result of relaxation processes in an associated receive coil, forming the basis for the image calculation.

It has been possible recently to miniaturize the magnetic field generating elements and the transmit and receive coils and integrate them in an imaging IVMRI sensor so that an intracorporeal or intervascular application of the MRI method (MRI=Magnetic Resonance Imaging) is possible, with the required static magnetic field advantageously being generated or applied within the patient's body. Such a concept is described for example in U.S. Pat. No. 6,600,319.

To this end a permanent magnet or electromagnet for generating a static magnetic field and a coil that is equally effective as a transmit and receive coil are integrated in the IVMRI sensor. The magnet generates field gradients of preferably 2 T/m up to 150 T/m in proximity to the vessel or organ to be examined. In proximity here means up to 20 mm from the magnet. Depending on the strength of the magnetic field radio waves in the frequency range from 2 MHz to 250 MHz can be decoupled by way of the coil to excite the surrounding body tissue. Higher static magnetic field strengths require higher excitation field frequencies. The coil advantageously also serves to receive the associated “response field” from the body tissue. In one alternative embodiment separate transmit and receive coils can be provided.

In contrast to conventional MRI systems the IVMRI sensor and the electronic switching circuits provided for signal processing and evaluation and the digital evaluation units are advantageously designed so that they can operate with high local field gradients even with a relatively non-homogeneous magnetic field and produce corresponding magnetic resonance images. Since in such conditions the received echo signals are characteristically influenced by the microscopic diffusion of water molecules in the examined tissue, generally an excellent representation and differentiation is allowed between different soft parts, e.g. between lipid layers and fibrous tissue. This is of particular interest precisely in the area of deployment of minimally invasive interventions provided for here.

As an alternative to the concept described here the static magnetic field can also be generated by external magnets. In contrast to conventional MRI the dynamic fields, i.e. the radio waves, are however also generated in an intervascular manner with this embodiment, in other words by a number of transmit and receive units disposed on the catheter.

According to a third advantageous variant an optical image sensor, optionally a CMOS, OCT, LCI, OFDI or NIR sensor is provided as the imaging sensor.

For example an optical semiconductor detector based on the known CMOS technology (CMOS=Complementary Metal Oxide Semiconductor) can be used to detect incident light. Such a CMOS sensor, also known as an “Active Pixel Sensor”, like the CCD sensors (CCD=Charge-Coupled Device) known primarily from the field of digital photography, is based on the internal photoelectric effect and as well as low power consumption also has the advantage that that it is particularly economical to produce. To illuminate the examination and treatment region with this imaging variant a suitable light source, e.g. an LED (LED=Light Emitting Diode) should be provided in the region of the catheter tip, being able to be supplied with electric current by way of an electric line guided through the hollow chamber of the catheter.

In one further variant the catheter can also be equipped with a sensor for optical coherence tomography (OCT=Optical Coherence Tomography).

Optical coherence tomography imaging provides high-resolution images, which reproduce in particular the structures in proximity to the vessel surface in a relatively exact manner. The principle of this method is based on light supplied from the catheter by way of an optical waveguide, preferably infrared light, being radiated into the vessel or onto a tissue structure, with the light reflected there being coupled back into the optical waveguide and fed to an evaluation facility. In the evaluation unit—as with a Michelson interferometer—the interference of the reflected light with the reference light is evaluated for image generation.

While conventional interferometric apparatuses preferably operate with laser light of defined wavelength, which has a relatively long optical coherence length, light sources with wide-band emission characteristics (“white light”) and with a relatively short coherence length of the emitted light are used with so-called LCI (LCI=Low Coherence Interferometry). Corresponding image sensors, which according to one advantageous embodiment of the invention are provided for use in the catheter, are described for example in US 2006/0103850.

In one advantageous modification an image sensor can also be provided, which is based on the so-called OFDI principle (OFDI=Optical Frequency Domain Imaging). The method is related to OCT but uses a wider frequency band. The operating principle is described in more detail for example in the publication “Optical frequency domain imaging with a rapidly swept laser in the 815-870 nm range”, H. Lim et al., Optics Express 5937, Vol. 14, No. 13.

Finally the catheter can also have an imaging sensor, which is based on so-called “Near-Infrared (NIR) Diffuse Reflectance Spectroscopy”. An NIR apparatus consists of a laser light source, a fiber-optic catheter and an automatic withdrawal apparatus. An NIR sensor is described for example in US 2003/0097048 A1.

Also combinations of at least two optical sensors of the type mentioned above can also be present.

A tabular summary sets out the strengths and weaknesses of the respective optical imaging methods (from ++=particularly good or suitable to −−=poor or unsuitable):

| Comparison of | Close | Distance | Blood |
|---|---|---|---|
| image sensors | resolution | resolution | penetration |
| Optical (CMOS) | + | + | − |
| OCT | ++ | − | −− |
| LCI | + | + | + |
| NIR | − | − | +/− |
| OFDI | ++ | − | + |

Since the spatial angle that can be detected or is to be viewed with the respective image sensor is generally limited, it is advantageous, particularly with the above-mentioned configuration with a radial viewing direction (in relation to the center axis of the catheter), if the imaging sensor is supported in such a manner that it can be rotated in relation to the catheter. This makes it possible to obtain a 360° all-round view without having to rotate the catheter itself in relation to its surroundings within the body. The (mechanical or electronic) rotation of the image sensor during its simultaneous retraction or advance advantageously allows 3D images or volume data records to be generated by means of suitable signal processing and image calculation methods known in principle from the prior art.

According to the invention the object is also achieved by a medical examination and treatment facility with a catheter arrangement according to one of the preceding embodiments, wherein the position identification element is connected to an imaging processing and playback facility outside the catheter and transmits information from the site of the minimally invasive intervention carried out with the aid of the catheter to this in real time.

A first position identification element here is advantageously part of a location systems and a second position identification element is configured as an imaging sensor, with a control facility being set up to activate the first and second position identification elements one after the other in temporal succession. To prevent the image processing and playback facility being influenced and the different magnetic fields of the first and second position identification elements being mutually influenced, the various units are synchronized and activated and read out in a temporally offset manner. For example in a first step the position of the catheter tip in the blood vessel is determined and then the imaging sensor is used to visualize the spatial region around the catheter tip or in a forward direction to monitor navigation of the intervention tool. This process is repeated a number of times as the catheter and intervention tool are guided to the blood dot.

According to the invention the object is also achieved by a method for minimally invasive intervention at a blood vessel in the brain, wherein a catheter arrangement with a catheter and an intervention tool for removing a blood clot from the blood vessel is guided to a region to be treated, with a position identification element being disposed in the region of the catheter tip, which is used to determine the position of the catheter tip in real time and with the catheter advance being monitored and the position of the intervention tool being checked.

The advantages and preferred embodiments listed in relation to the catheter arrangement apply appropriately to the medical examination and treatment facility and to the method for minimally invasive intervention.

An expedient workflow for the deployment of the catheter arrangement with the integrated position identification element is for example as follows:

1. Positioning of the patient on the treatment table,
2. Any preparatory x-ray examination and/or extracorporeal ultrasound examination,
3. Insertion of the catheter by way of a vein,
4. Guidance of the catheter based on integrated imaging to the region to be treated in the brain,
5. Outward movement and extension of the intervention tool,
6. Inflation of a securing balloon for the catheter
7. Retraction of the intervention tool with the thrombus enclosed therein,
8. Check, if required, with the imaging element, whether the thrombus has been completely removed,
9. Removal of the catheter,
10. Additional final x-ray check, if required, and/or extracorporeal ultrasound examination,
11. Transfer of patient.

In the case of IVMRI imaging, based on gadolinium for example, or for ultrasound imaging based on sulfur hexafluoride it can be expedient to apply a contrast agent at the observation site.

To summarize, the catheter arrangement described here primarily allows optimization of the medical workflows during a minimally invasive intervention to remove a blood clot from the brain. Such interventions can be completed with a higher degree of patient safety and at the same time more quickly and in a more patient-friendly manner than before.

### BRIEF DESCRIPTION OF THE DRAWINGS

An exemplary embodiment of the invention is described in more detail below with reference to the schematic and highly simplified diagrams in a drawing, in which:

FIG. 1 shows a catheter arrangement with a retracted intervention tool with a closed spiral at its tip,

FIG. 2 shows the catheter arrangement according to FIG. 1 with an intervention tool moved out with an opened out spiral,

FIG. 3 shows a second variant of a catheter arrangement with an optical sensor guided by means of a catheter and a transparent window for the optical sensor disposed on the end face of the catheter,

FIG. 4 shows a third variant of a catheter arrangement with an optical sensor guided by means of a catheter and a transparent window for the optical sensor disposed on the periphery of the catheter,

FIG. 5 shows a fourth variant of a catheter arrangement with an optical sensor guided by means of a catheter without a transparent window,

FIG. 6 shows a fifth variant of a catheter arrangement with an optical sensor guided by means of a catheter and a location system on the catheter,

FIG. 7 shows a sixth variant of a catheter arrangement with an optical sensor guided by means of a catheter, a transparent window on the catheter and a location system on the catheter,

FIG. 8 shows a CMOS sensor for imaging in a radial direction,

FIG. 9 shows a CMOS sensor for forward imaging,

FIG. 10 shows an OCT sensor for imaging in a radial direction,

FIG. 11 shows an OCT sensor for forward imaging,

FIG. 12 shows an IVMRI sensor for imaging in a radial direction,

FIG. 13 shows an IVMRI sensor for forward imaging,

FIG. 14 shows an IVUS sensor for imaging in a radial direction combined with forward imaging,

FIG. 15 shows an enlarged front view of the IVUS sensor according to FIG. 16, and

FIG. 16 shows a diagram of synchronized reading out of a number of sensors.

Parts with identical action are shown with the same reference characters in the figures.

### DETAILED DESCRIPTION OF THE INVENTION

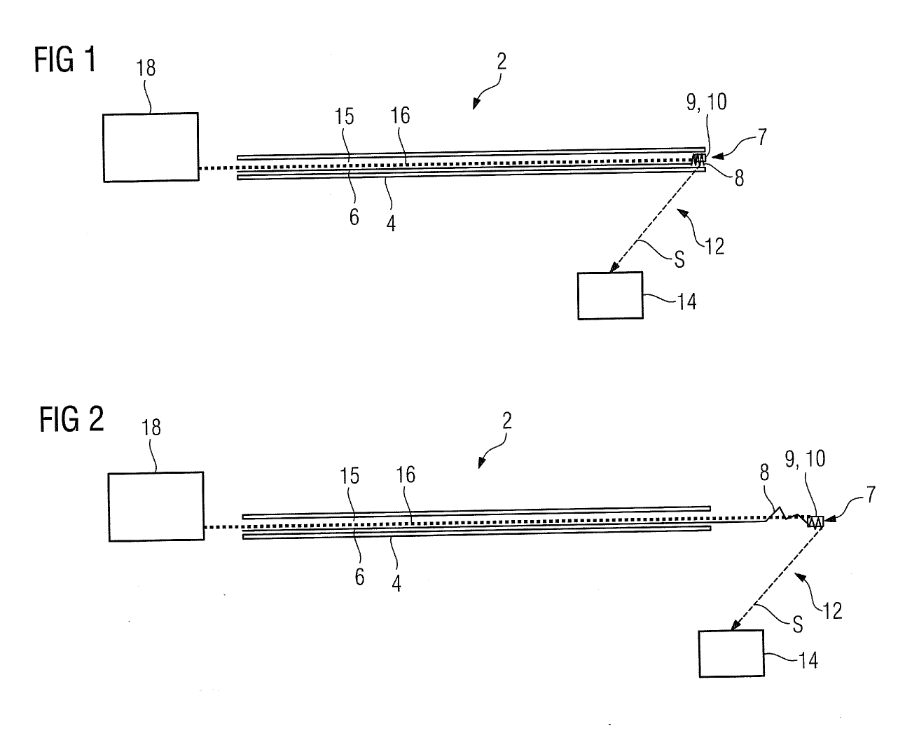

FIG. 1 and FIG. 2 show a catheter arrangement 2 for a minimally invasive surgical intervention, comprising a catheter 4 and an intervention tool 6. The intervention tool 6 is configured to remove a blood clot from a blood vessel in the brain of a patient and is guided in the blood vessel by means of the catheter 4 until it reaches the blood clot. The intervention tool 6 has an element for trapping the blood clot in the region of its tip 7, in this instance an opening spiral 8 (see FIG. 2).

For optimal and long-lasting healing success and to minimize any intervention risks, it is important for it to be possible to observe the catheter arrangement 2 and its local surroundings within the body with the best resolution possible during its advance through a blood vessel so that prompt and fine position corrections can be made. In particular it is important for the intervention tool 6 to be positioned as precisely as possible in the correct position for the respective intervention. Until now such monitoring has generally been achieved by means of angiographic x-ray control.

For better quality monitoring without using ionizing x-ray radiation the catheter arrangement 2 according to FIG. 1 and FIG. 2 has a position identification element 9 at the tip 7 of the intervention tool 6, which in this exemplary embodiment is part of an electromagnetic location systems 12. The position identification element 9 here is a position transmitter 10, which communicates with an external position receiver 14 in proximity to the patient, the location signal S of the position transmitter 10 being shown by the arrow 5. Alternatively the position identification element 9 can be a position receiver, which receives location signals from an external position transmitter. Regardless of whether the position identification element 9 is a transmitter or a receiver, it is configured to emit and/or receive signals for locating the tip 7 of the intervention tool 6 in all three spatial directions. The location of the position identification element 9 allows the position of the tip 7 of the intervention tool 6 to be located, not only on the way to the blood clot but also during the intervention.

The catheter 4 encloses a cylindrical hollow catheter chamber 15 (also referred to as the lumen), through which signal lines 16 are passed, to connect the electromagnetic position transmitter 10 to a control unit 18 for data purposes.

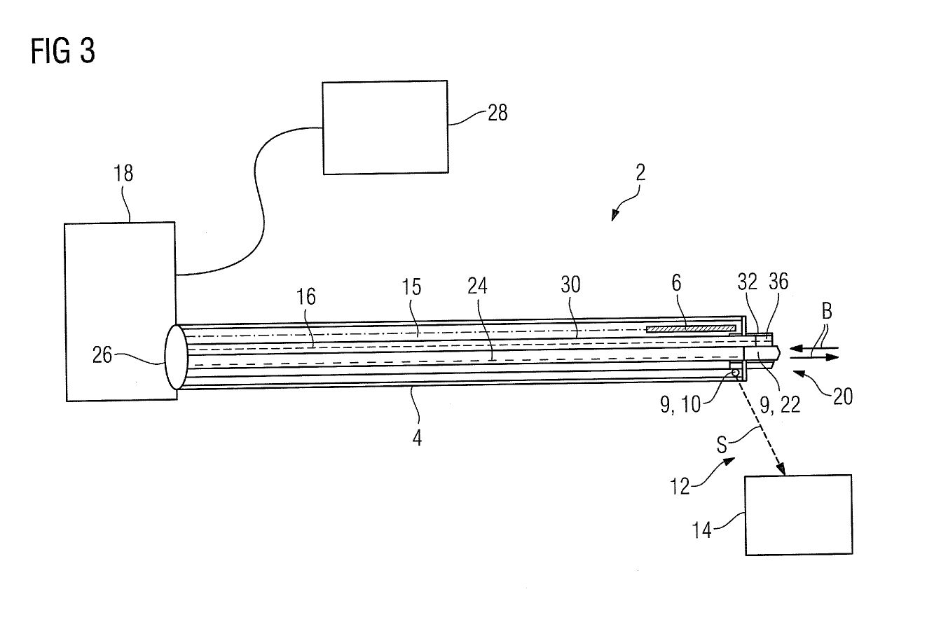

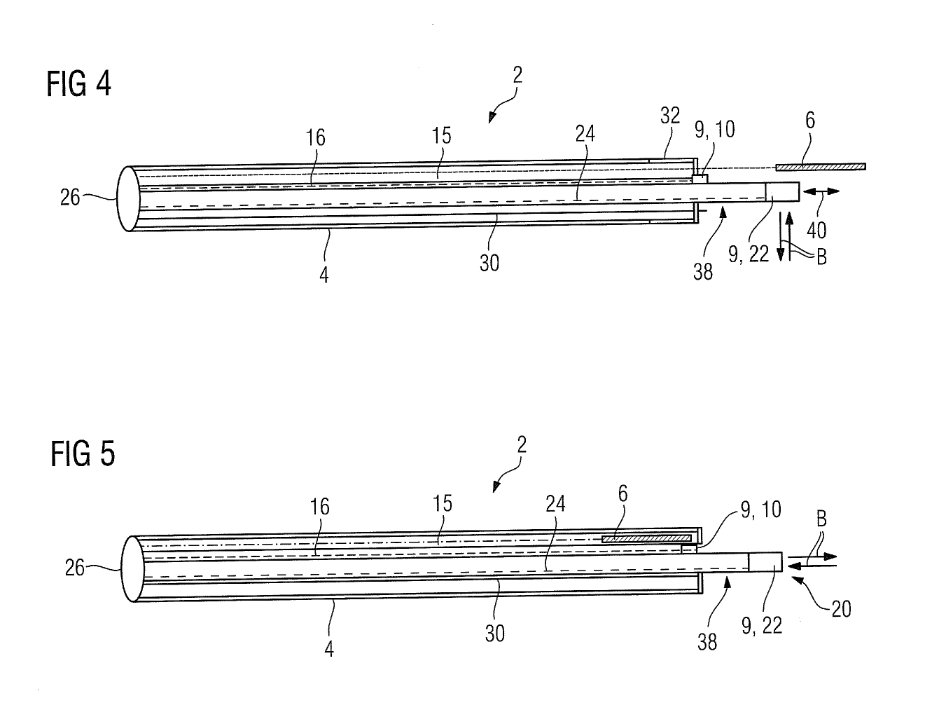

The catheter arrangement 2 shown in FIG. 3 likewise comprises a flexible catheter 4 for inserting the intervention tool 6 into a blood vessel (not shown in more detail). In FIG. 3 the intervention tool 6 is shown in its transport position, being fully retracted or drawn into the catheter 4. To carry out the intervention the intervention tool 6 is moved out of the catheter 4 in the proximal direction and thus moved into a treatment position (see FIG. 4).

In addition to the electromagnetic location system 12 the catheter arrangement 2 according to FIG. 3 is also equipped with a second position identification element 9, an imaging sensor 22, which is disposed to the side of the intervention tool 6 in the region of the catheter tip 20. Depending on the sensor type and other details of its configuration the “field of vision” B of the sensor 22 is preferably directed radially outward (to the surrounding vessel wall, not shown here) and/or in the proximal direction forward (i.e. in the direction of advance of the catheter 4), as shown symbolically by the arrows B.

The imaging sensor 22 can be for example an optical sensor, an acoustic (ultrasound) sensor or a sensor based on the magnetic resonance principle. The signal and supply lines 24 required for its operation and for transmitting the recorded image data are guided in the interior of the catheter jacket 4 to a connecting coupling 26 disposed at the (distal) end of the catheter 2 facing away from the body. The imaging electronic components of the catheter arrangement 2 can be connected electrically to a signal interface (only shown schematically) by way of the connecting coupling 26, said interface corresponding to the control unit 18 according to FIG. 1 and FIG. 2 and being connected for its part to an external image processing and playback facility 28. A monitor (not shown in more detail) serves to play back the “live images” from the treatment site recorded in an intervascular manner by the imaging sensor 22 and in some instances then processed computationally.

In order to be able to rotate the imaging sensor 22 about its own axis within the fixed catheter 4, a rotatable drive shaft can also be disposed in the hollow catheter chamber 6 but this is not shown in detail in FIG. 3. The imaging sensor 22, the signal lines 24 and optionally the drive shaft can be combined to form a compact unit in the manner of a micro or inner catheter disposed within the outer catheter jacket 4 and be enclosed by an inner protective jacket 30. In particular when interferometric imaging methods are used, optical waveguides can also be present within the inner catheter, to pass incident and outward light beams to an externally positioned interferometer unit or the like that can be connected by way of the connecting coupling 26. In the region of the imaging sensor 22 the inner protective jacket 30 and/or the catheter 4 expediently has a transparent window 32 for the respective imaging method, optionally also an optical lens.

Also one or more lines (not shown here) can (optionally) be provided for a rinse liquid or contrast agent, which can be injected into the region to be examined/treated by way of an exit opening 36 at the proximal end of the catheter 4 disposed in proximity to the imaging sensor 22.

In the exemplary embodiment according to FIG. 3 in the region of the catheter tip 20 the position transmitter 10 is disposed directly adjacent to the imaging sensor 22, which interacts with the position receiver 14 disposed outside the body of the patient according to the transmitter/receiver principle to allow precise location of the catheter tip 20 by identifying the coordinates of said catheter tip 20. The position data thus obtained can be fed for example to the image processing and playback facility 28 and be taken into account during image reconstruction, specifically during artifact correction. The signal lines 16 for the position transmitter 10 can likewise be guided within the (inner) protective jacket 30 essentially parallel to the signal lines 24 of the imaging sensor 22.

FIG. 4 to FIG. 7 respectively show structural modifications of the catheter arrangement 2.

Thus for example in FIG. 4 an inner part 38 supporting the imaging sensor 22 can be moved forward (in the proximal direction) in relation to the catheter 4 from a retracted position corresponding to the position in FIG. 3 to the more forward position shown here and vice versa (shown by the double arrow 40). In other words the imaging sensor 22 can be pushed out in a forward direction if required beyond the proximal end of the catheter 4, where it has an unrestricted view, in particular of the intervention tool 6, which has also been moved out of the catheter 4 in FIG. 4. The outward/inward movement of the intervention tool 6 and of the imaging sensor 22 can preferably be effected independently of one another.

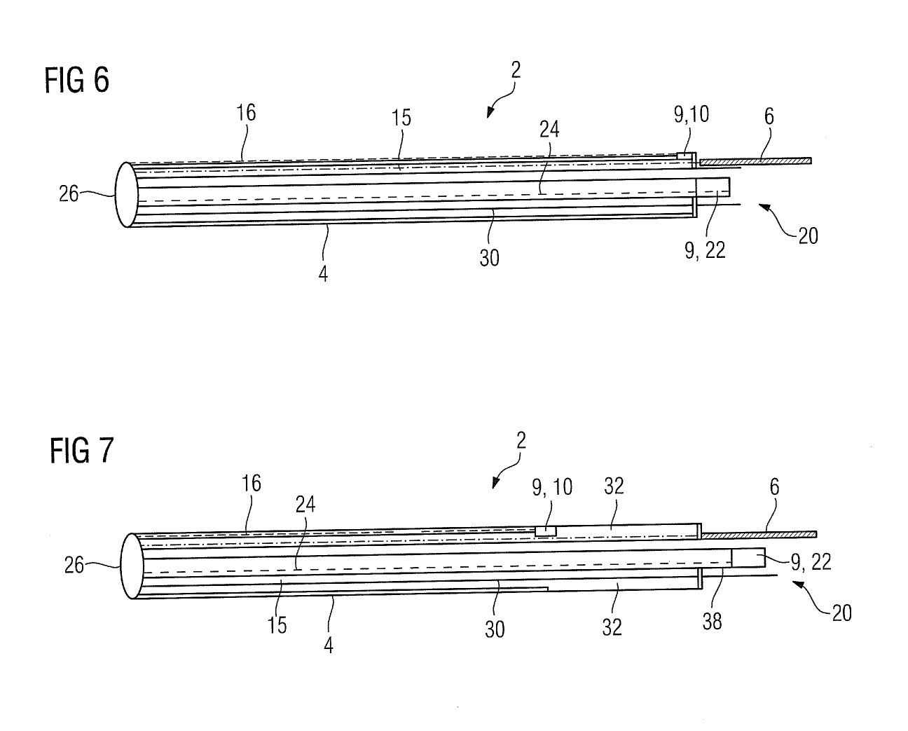

The embodiment according to FIG. 5 corresponds essentially to the one in FIG. 3 or FIG. 4, but here there is no transparent window on the catheter jacket 4. The embodiment according to FIG. 6 is also held like those described above but in this variant the position transmitter 10 is disposed outside on the catheter 4. With the variant according to FIG. 7 finally the displacement path of the imaging sensor 22 in a longitudinal direction in the catheter 4 is enlarged. The position transmitter 10 here is positioned further toward the end of the catheter 4 facing away from the body and the transparent window 32 is enlarged.

The catheter arrangements 2 described above are deployed in the following manner for a patient suffering an ischemic stroke: at the start of the treatment the patient undergoes an x-ray examination. The x-ray examination can be a fluoroscopy for example and/or an angiography examination using a contrast agent, providing image data, in particular for the 3D reconstruction of the blood vessels. The catheter 4 is used to insert the intervention tool 6 by way of a vein into the body of the patient. Navigation of the catheter 4 is assisted here by the location system 12, with the aid of which the position of the catheter tip 20 in the blood vessel is known at any time. The imaging sensor 22 also sends images from the interior of the blood vessel around the catheter tip 20. The position of the position transmitter 10 at least is calibrated before the treatment using the image data obtained from the x-ray examination, so that the movement of the catheter arrangement 2 in the blood vessel can be tracked by overlaying with the x-ray images.

During insertion of the intervention tool 6 up to the blood clot it is in a retracted position in the catheter 4 and the spiral 8 remains closed. When the blood clot is reached, the intervention tool 6 is moved out of the catheter 4 and passed through the blood clot. The spiral 8 does not open out until it is behind the blood clot and when the intervention tool 6 is retracted in the direction of the catheter 4 the blood clot becomes trapped in the spiral 8 and is carried along with it.

The catheter 4 is in particular a guide catheter, which has an inflatable balloon downstream. The balloon is inflated once the blood clot has been trapped, so that the catheter 4 sits thinly in the vessel, when the intervention tool 6 draws out the blood clot.

The intervention tool 6 is retracted together with the enclosed blood clot in the direction of the guide catheter 4, the location system 12 and imaging sensor 22 still being used to locate the position of the catheter tip 20 and the interior of the blood vessel still being visualized. When the still extended spiral 8 together with the enclosed blood clot reach the guide catheter, they are retracted into it, so that the blood clot is no longer exposed to the blood flow in the vessel. The blood clot is removed by withdrawing the guide catheter 4 from the body of the patient. Finally a further x-ray examination can be carried out to verify the success of the treatment.

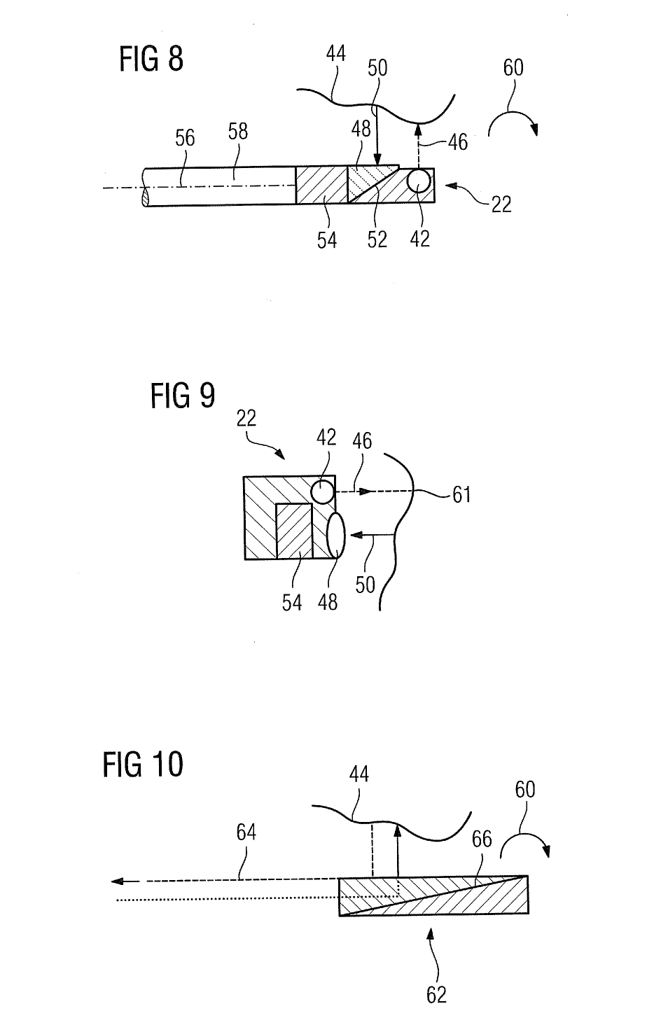

In the diagram of the detail according to FIG. 8 the region of the catheter tip 20 with the imaging sensor 22 is shown enlarged, with a CMOS-based optical sensor being used in the variant illustrated here. A light source 42, in this instance a high-power micro LED, illuminates the vessel wall 44 enclosing the imaging sensor 22 (emitted light 46). Light 50 reflected off the vessel wall 44 passes through a lens 48 to a reflective mirror 52 (or even for example a prism with a similar mode of operation or beam guidance) and from there to the actual CMOS image detector 54. The arrangement according to FIG. 8 is thus configured for a radial viewing direction (in relation to the center axis 56 of the catheter 2). A rotational movement brought about with the aid of a drive shaft 58 about the center axis 56, shown by the arrow 60, allows the full lateral 360° field of vision to be covered.

As an alternative FIG. 9 shows an example of a configuration of light source 42, lens 48 and CMOS detector 54, which allows forward observation, which is particularly useful when the catheter 2 is being advanced through the blood vessels. An obstacle 61 in the forward direction, which might impede the further advance, can thus be identified. The two variants according to FIG. 8 and FIG. 9 can optionally also be combined, to provide a particularly comprehensive field of vision in practically all directions.

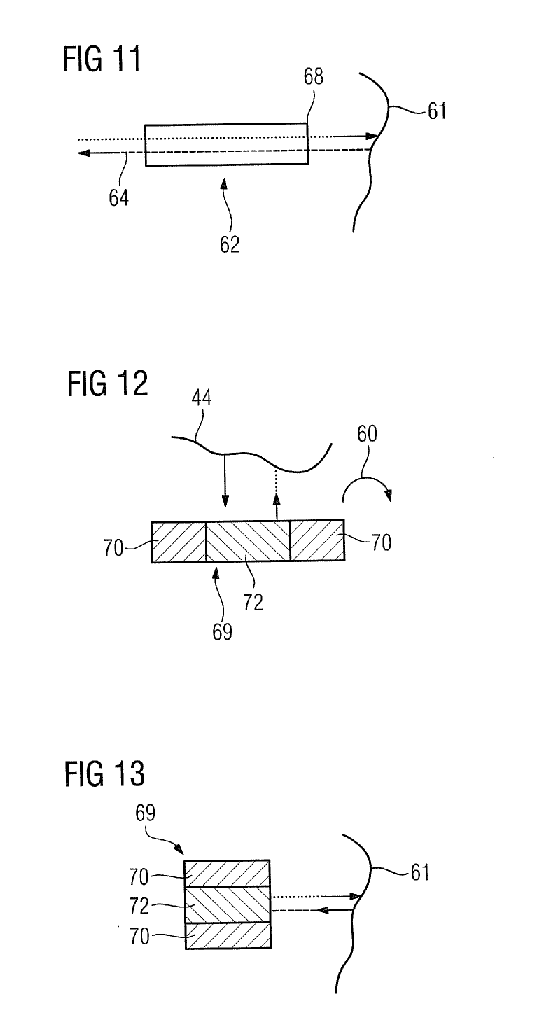

The above-mentioned observation directions, namely radial/lateral and forward, can also be realized with other sensor types. For example FIG. 10 shows a configuration of an OCT or LCI sensor head 62 for radial emission and receiving and FIG. 11 for forward emission and receiving. More specifically the reference character 62 only designates the sensor part or sensor head responsible for coupling the light into and out of the optical waveguide 64; actual interferometric evaluation and image generation take place outside the catheter arrangement 2. The beam path of the coupled out and reflected portion of the light beams influenced by the reflective mirror 66 and the lens 68 is shown in each instance.

Similarly an IVMRI sensor or IVUS sensor can also be configured either for radial or forward emission/receiving, as shown schematically in FIG. 12 and FIG. 13 for an IVMRI sensor 69 with permanent magnets 70 for the static magnetic field and transmit/receive coils 72.

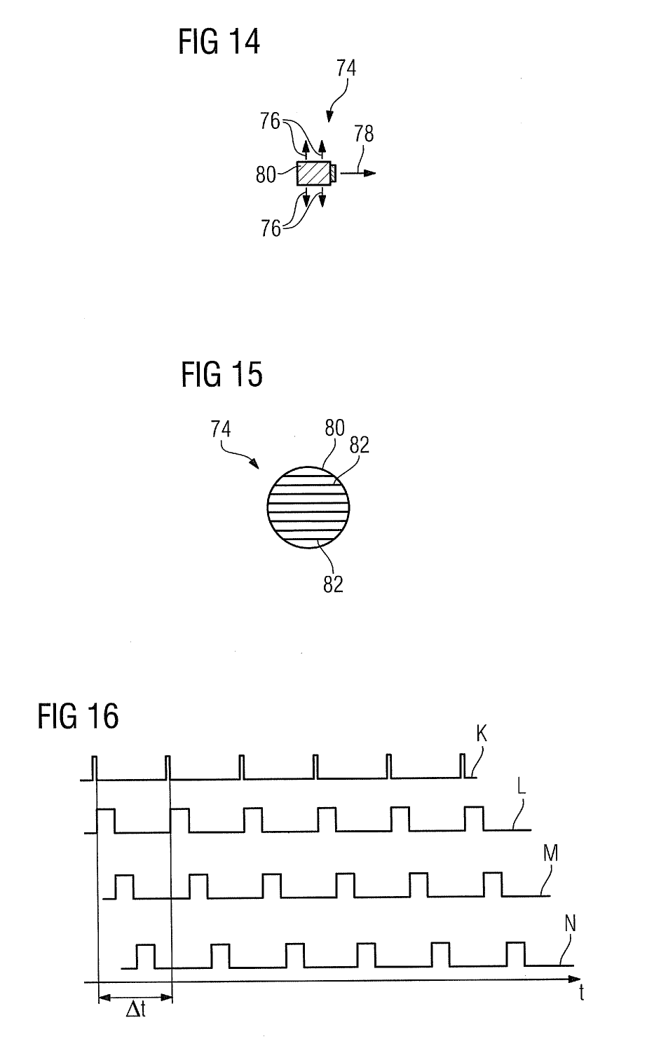

In the case of lateral emission/receiving it can be advantageous in particular in the case of ultrasound sensors to provide an array of ultrasound sensor elements with different “viewing directions” instead of a single rotating sensor. Such an NUS sensor 74 is shown in FIG. 14 and FIG. 15. The IVUS sensor 74 is configured both for imaging in a radial direction, shown by arrows 76, and for imaging in a forward direction 78. As can be seen from the enlarged representation of the end face of the IVUS sensor 74 according to FIG. 15, a number of line-type ultrasound sensor elements 82 are disposed parallel to one another on a sensor unit 80. The ultrasound elements 82 are activated cyclically, i.e. excited and interrogated cyclically, by way of a multiplexer (not shown here).

Since many units required for the intervention are operated with electrical power, they generate magnetic fields, which can influence one another (e.g. in the case of an electromagnetic location system 12 in combination with an IVMRI sensor 69). To avoid this, the various units are activated with a temporal offset by the control unit 18 and their signals are read out temporally one after the other. Such synchronized activation over time t is shown in FIG. 16. The clock signal K indicates a system clock by way of example. This is predetermined for example by: the electromagnetic location system 12, the control unit 18, the image processing and playback facility 28 or an image system of the x-ray system. L designates the signal 4 of the electromagnetic location system 12 which is operated pulsed. As soon as the signal L of the location system 12 is read out, the IVMRI sensor 69 is also read out (the curve M), to visualize an image of the surroundings around and in front of the catheter tip 20. Finally if an ECG device or a respirator is used, the signal of which is shown by the curve N, this is likewise actuated briefly. All this takes place within a period Δt, which is for example in the region of milliseconds, in particular between 10 ms and 3000 ms.

1.-16. (canceled)

17. A catheter arrangement for inserting into a blood vessel, comprising:
a catheter comprising a catheter tip;
an intervention tool arranged in the catheter that is guided to remove a blood clot from the blood vessel;
a device arranged on the intervention tool that traps a blood clot in a region of a tip of the intervention tool; and
a position identification element disposed in a region of the catheter tip that determines a position of the catheter tip in real time.

18. The catheter arrangement as claimed in claim 17, wherein the position identification element is disposed at the tip of the intervention tool.

19. The catheter arrangement as claimed in claim 17, wherein the position identification element is a part of a location system comprising a position transmitter and a position receiver.

20. The catheter arrangement as claimed in claim 19, wherein the position identification element is the position transmitter or the position receiver.

21. The catheter arrangement as claimed in claim 19, wherein the location system is an electromagnetic location system or an ultrasound system.

22. The catheter arrangement as claimed in claim 17, wherein the position identification element is an imaging sensor and a field of vision of the imaging sensor covers a spatial region around the region of the catheter tip or a spatial region in front of the region of the catheter tip.

23. The catheter arrangement as claimed in claim 22, wherein the region of the catheter tip comprises a transparent window.

24. The catheter arrangement as claimed in claim 22, wherein the imaging sensor is moved outward in relation to the catheter.

25. The catheter arrangement as claimed in claim 22, wherein the imaging sensor is rotated in relation to the catheter.

26. The catheter arrangement as claimed in claim 22, wherein the imaging sensor is selected form the group consisting of: an ultrasound sensor, a magnetic resonance sensor, and an optical image sensor.

27. The catheter arrangement as claimed in claim 26, wherein the optical image sensor is selected from the group consisting of: a complementary metal oxide semiconductor sensor, an optical coherence tomography sensor, a low coherence interferometry sensor, an optical frequency domain imaging sensor, and a near-infrared sensor.

28. The catheter arrangement as claimed in claim 17, wherein the device is a spiral.

29. A medical examination and treatment device, comprising:
a catheter comprising a catheter tip;
an intervention tool arranged in the catheter that is guided to remove a blood clot from the blood vessel;
a device arranged on the intervention tool that traps a blood clot in a region of a tip of the intervention tool; and
a position identification element disposed in a region of the catheter tip and connected to an image processing and playback device outside the catheter that transmits information from a site of a minimally invasive intervention to the image processing and playback device in a real time and determines a position of the catheter tip in the real time.

30. The medical examination and treatment device as claimed in claim 29, wherein the position identification element is a part of a location system.

31. The medical examination and treatment device as claimed in claim 29, further comprising a second position identification element that is an imaging sensor.

32. The medical examination and treatment device as claimed in claim 31, further comprising a control device that activates the position identification element and the second position identification element one after the other in a temporal succession.

33. A method for minimally invasive intervention at a blood vessel in a brain, comprising:
providing a catheter comprising a catheter tip;
arranging an intervention tool in the catheter;
guiding the intervention tool by the catheter;
removing a blood clot from the blood vessel by the intervention tool;
disposing a position identification element in a region of the catheter tip;
determining a position of the catheter tip in real time by the position identification element;
monitoring the catheter based on the determination; and
checking a position of the intervention tool based on the determination.
